(19) United States
(12) Patent Application Publication
(10) Pub. No.: US 2020/0022742 A1
(43) Pub. Date: Jan. 23, 2020

(54) EPIPHYSEAL FRACTURE FIXATION DEVICES AND METHODS OF USE THEREOF

(71) Applicant: WAKE FOREST UNIVERSITY HEALTH SCIENCES, Winston Salem, NC (US)

(72) Inventors: ZHONGYU JOHN LI, Kernersville, NC (US); PHILIP BROWN, Winston Salem, NC (US)

(21) Appl. No.: 16/496,243

(22) PCT Filed: Mar. 20, 2018
(86) PCT No.: PCT/US18/23357

§ 371 Date: Sep. 20, 2019

Related U.S. Application Data
(60) Provisional application No. 62/473,520, filed on Mar. 20, 2017.

(51) Int. Cl. A61B 17/72

(52) U.S. Cl. CPC A61B 17/7283; A61B 2017/564

(57) ABSTRACT

Intramedullary nails are provided for the fixation of epiphyseal fragments in a long bone of a patient in need thereof. The intramedullary nails have a cap portion that allows for fixation of the epiphyseal bone fragments thereto, and an intramedullary shaft for insertion into the medullary canal of the long bone. The intramedullary nails can provide for fixation of comminuted fractures that are difficult to treat with conventional methods. In some aspects, the patient is a human patient and the fracture is a Mason type II radial head fracture or a Mason type III radial head fracture. Methods of fixation of epiphyseal fractures using the intramedullary nails of the present disclosure are also described.

### CROSS-REFERENCE TO RELATED APPLICATIONS

This application claims priority to, and the benefit of, co-pending U.S. provisional application entitled “EPIPHYSEAL FRACTURE FIXATION DEVICES AND METHODS OF USE THEREOF” having Ser. No. 62/473,520, filed Mar. 20, 2017, the contents of which are incorporated by reference in their entirety.

### TECHNICAL FIELD

The present disclosure generally relates to bone fixation devices and methods of use thereof.

### BACKGROUND

The skeletal systems of humans and a variety of other mammals include many long bones that extend from a central torso. For example, in humans, these long bones include the femur, fibula, tibia, humerus, radius and ulna. Long bones, especially the femur and tibia, are subjected to most of the load during daily activities and they are crucial for skeletal mobility. Generally speaking, these long bones are defined by a long central shaft region called the diaphysis having a hollow central region (the medullary cavity) for containing the bone marrow and enclosed on each end by the epiphysis (head) of the long bone. Between the epiphysis and the diaphysis is the metaphysis, which contains the growth plate, the part of the bone that grows during childhood and as it grows, ossifies near the diaphysis and the epiphyses.

These long bones are particularly exposed to trauma from accidents, and, as such, may be fractured during a trauma. These fractures can be particularly difficult to treat when occurring in the epiphyseal region (head) of the long bone. Automobile accidents, for instance, are a common cause of trauma to long bones. In particular, the femur and tibia frequently fracture when the area around the knee is subjected to a frontal automobile accident. Another common area of fractures in humans includes the elbow region, where radial head/neck fractures are the most common. Radial head and neck fractures are estimated to occur at an incidence rate of 2.8 per 10,000 per year in the general population. They can occur in isolation or associated with elbow dislocation, or other elbow and forearm fractures.

Mason type II or III radial head fractures often require an open reduction internal fixation (ORIF), radial head resection or replacement arthroplasty. Studies showed that radial head fractures treated by ORIF had less pain, better range of motion (ROM) and strength than those treated with radial head resection. The late complications of arthroplasty, such as implant loosening remain a major concern after radial head arthroplasty (Walters et al. Clin Orthop Relat Res (2014) 472: 2128). Standard treatments include k-wires, cortical screws, headless screws, plate, and specially designed radial head plates. Although, k-wire or screw fixations may be adequate for simple radial head fractures, comminuted radial head fractures or radial neck fractures frequently require plate fixation. This requires a large number of different purpose specific designed radial head plates for clinical use. There is not a simple and universal radial head plate that readily fits all radial heads. Furthermore, existing treatments typically result in prominent hardware at or near the radial head causing soft tissue irritation and elbow stiffness are common after radial head ORIF. The complications often require a second surgery for hardware removal and elbow contracture release.

There remains a need for improved methods of fixation in epiphyseal fractures, especially in radial head fractures, that overcome the aforementioned deficiencies.

### SUMMARY

A variety of epiphyseal fracture fixation devices and methods are provided that overcome one or more of the aforementioned deficiencies. The epiphyseal fracture fixation devices include an intramedullary nail for fixation of an epiphyseal fracture in a long bone of a patient in need thereof. The intramedullary nail can include an intramedullary shaft for securing the nail in the intramedullary canal and a cap portion for securing one or more epiphyseal bone fragments. The intramedullary nail can be made partially or entirely from a thermoplastic polymer, for example a polyethylene, a polymethylmethacrylate, a polyurethane, a polysulfone, a polyetherimide, a polyimide, an ultra-high molecular weight polyethylene (UHMWPE), a cross-linked UHMWPE, a polyaryletherketone, a copolymer thereof, or a blend thereof. Polyaryletherketones can include poly(aryl-ether-ether-ketone) (PEEK), polyetherketoneketone (PEKK), or poly(aryl-ether-ketone-ether-ketoneketone) (PEKEKK).

In some aspects, the intramedullary nail includes an intramedullary shaft a proximal end and a distal end, a cap portion at the proximal end and configured for attaching thereto one or more epiphyseal bone fragments from the epiphyseal fracture, and a tip portion at the distal end and configured for insertion into the medullary canal of the long bone. The intramedullary nail can have a longitudinal axis extending the length of the intramedullary shaft.

The intramedullary nail can include the cap portion configured for attaching thereto the one or more epiphyseal bone fragments. The cap portion can be configured to receive one or more self-drilling screws for attaching the one or more epiphyseal bone fragments to the cap portion. The cap portion can have an outer surface substantially parallel to the longitudinal axis of the intramedullary shaft, and the outer surface can have a plurality of grooves running longitudinally along the outer surface. The plurality of grooves on the outer surface of the cap can be configured to receive one or more self-drilling screws for attaching the one or more epiphyseal bone fragments to the outer surface of the cap portion. The grooves on the outer surface of the cap can have a depth of about 0.3 mm to about 0.9 mm. The plurality of grooves on the outer surface of the cap can include about 15 to 25 grooves equally spaced circumferentially along the outer surface of the cap.

The cap portion can include an upper surface having an opening dimensioned to receive a tip of a tool for tightening the intramedullary nail into the medullary canal. The cap portion can have a height in the longitudinal direction of about 6 mm to about 10 mm. The cap portion can have an outer diameter of about 6 mm to about 15 mm

The intramedullary shaft of the intramedullary nail can have an outer surface substantially parallel to the longitudinal axis of the intramedullary shaft, and the outer surface can have a plurality of grooves running longitudinally along the outer surface. The plurality of grooves on the outer surface of the intramedullary shaft can include about 2 to 8 grooves equally spaced circumferentially along the outer surface of the intramedullary shaft. The grooves on the outer surface of the intramedullary shaft can have a depth of about 0.3 mm to about 1.5 mm. The intramedullary shaft can have an outer surface having a plurality of threads on the outer surface. The threads can be female threads or male threads. The threads can have any thread form, such as a square thread form, a triangular thread form, a trapezoidal thread form, or a circular thread form. The plurality of threads can have a number of thread starts from about 2 to 8. The threads can have an outer diameter of about 1 mm to about 2 mm. The threads can have a height of about 0.5 mm to about 3 mm. The intramedullary shaft can have an outer surface substantially parallel to the longitudinal axis of the intramedullary shaft, and the outer surface can be a broached surface.

The intramedullary shaft can have one or more void regions sized and positioned to contain one or more shavings from a self-drilling screw being inserted into the intramedullary shaft. The intramedullary shaft can have about 1 to 3 void regions located along the longitudinal axis of the intramedullary shaft. The intramedullary shaft can have a height in the longitudinal direction of about 15 mm to about 25 mm. The intramedullary shaft can have an outer diameter of about 4 mm to about 8 mm.

The intramedullary nails can be used to repair an epiphyseal fracture in a patient in need thereof. The patient can be a human patient or a non-human patient such as a dog, cat, horse, cow, sheep, or a goat. The intramedullary nail can, in principal, be used in any long bone in a patient in need thereof. The long bone can be a femur, a fibula, a tibia, a humerus, a radius, or an ulna. In some aspects, the patient is a human patient and the long bone is a radius.

Methods of fixation of an epiphyseal fracture in a long bone of a patient in need thereof are also provided. The methods can include attaching one or more epiphyseal bone fragments to a cap portion on a proximal end of an intramedullary nail, and inserting a distal end of the intramedullary nail into a medullary canal of the long bone. The methods can include attaching one or more epiphyseal bone fragments to the cap portion of an intramedullary nail described herein, and inserting the distal end of the intramedullary nail into a medullary canal of the long bone.

The methods can include inserting one or more self-drilling screws through an outer surface of the diaphysis and into the intramedullary shaft of the intramedullary nail to secure the intramedullary nail having the one or more epiphyseal bone fragments attached thereto. The self-drilling screw(s) can help to secure the intramedullary nail in the intramedullary shaft and prevent twisting or rotation of the intramedullary nail in the intramedullary shaft. The methods can also include applying a bone cement such as polymethyl methacrylate to the intramedullary shaft prior to inserting the distal end of the intramedullary nail into the medullary canal.

The methods can include repairing a variety of epiphyseal fractures. In some instances, one or more epiphyseal bone fragments of the fracture include two or more bone fragments. The epiphyseal fracture can be a Salter Harris type 1 fracture, a Salter Harris type 2 fracture, a Salter Harris type 3 facture, or a Salter Harris type 4 fracture. The fracture can be comminuted. In some aspects, the patient is a human and the long bone is a radius, and the epiphyseal fracture is a Mason type II radial head fracture or a Mason type III radial head fracture

Other systems, methods, features, and advantages of the intramedullary nails and methods of use thereof will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1E depict an exemplary embodiment of an intramedullary nail of the instant disclosure. FIG. 1A and FIG. 1C are perspective views of the intramedullary nail. FIG. 1B is a side elevation view of the intramedullary nail. FIG. 1D is a top plan view of the intramedullary nail. FIG. 1E is a bottom plan view of the intramedullary nail.

FIGS. 2A-2D depict an exemplary embodiment of an intramedullary nail of the instant disclosure. FIG. 2A is a side elevation view of the intramedullary nail. FIG. 2B is a sectional view along the line 1-1 in FIG. 2A. FIG. 2C is a top plan view of the intramedullary nail. FIG. 2D is a bottom plan view of the intramedullary nail.

FIGS. 3A-3B depict an exemplary embodiment of an intramedullary nail of the instant disclosure. FIG. 3A is a top plan view of the intramedullary nail. FIG. 3B is a sectional view along the line 2-2 in FIG. 3A.

FIGS. 4A-4F depict an exemplary embodiment of an intramedullary nail of the instant disclosure. FIG. 4A is a side elevation view of the intramedullary nail. FIG. 4B is a sectional view along the line 3-3 in FIG. 4A. FIG. 4C is a sectional view along the line 4-4 in FIG. 4A. FIG. 4D is a sectional view along the line 5-5 in FIG. 4A. FIG. 4E is a sectional view along the line 6-6 in FIG. 4A. FIG. 4F is a sectional view along the line 7-7 in FIG. 4A.

FIGS. 5A-5F depict an exemplary embodiment of an intramedullary nail of the instant disclosure. FIG. 5A and FIG. 5B are perspective views of the intramedullary nail from above and below respectively. FIG. 5C is a side elevation view of the intramedullary nail. FIG. 5D is a sectional view along the line 5-5 in FIG. 5C. FIG. 5E is a top plan view of the intramedullary nail. FIG. 5F is a bottom plan view of the intramedullary nail.

FIGS. 6A-6F depict an exemplary embodiment of an intramedullary nail of the instant disclosure. FIG. 6A and FIG. 6B are perspective views of the intramedullary nail from above and below respectively. FIG. 6C is a side elevation view of the intramedullary nail. FIG. 6D is a sectional view along the line 6-6 in FIG. 6C. FIG. 6E is a top plan view of the intramedullary nail. FIG. 6F is a bottom plan view of the intramedullary nail.

### DETAILED DESCRIPTION

In various aspects, intramedullary nails and methods of use thereof are provided. The intramedullary nails can be used for the fixation of an epiphyseal fracture in a long bone of a patient in need thereof. In various aspects, the use of the intramedullary nails of the present disclosure can lead to one or more of an improved rate of healing, improved comfort for the patient, decreased soft tissue irritation for the patient, and decreased joint stiffness for the patient as compared to standard treatments referenced above. In addition, in a number of aspects, the use of the intramedullary nails of the instant disclosure can lead to a reduced incidence of follow-on surgeries for hardware removal or elbow contracture release. In certain embodiments, the long bone is a radius and the fracture is a Mason type II or Mason type III radial head fracture.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting. The skilled artisan will recognize many variants and adaptations of the embodiments described herein. These variants and adaptations are intended to be included in the teachings of this disclosure and to be encompassed by the claims herein.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Embodiments of the present disclosure will employ, unless otherwise indicated, techniques of materials and device engineering as well as surgical and orthopedic techniques and the like, which are within the skill of the art. Such techniques are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

Unless otherwise specified herein, as used herein “about,” “approximately,” and the like, when used in connection with a numerical variable, can refer to the value of the variable and to all values of the variable that are within the experimental error (e.g., within the 95% confidence interval for the mean) or within ±10% of the indicated value, whichever is greater.

Intramedullary Nails

A variety of intramedullary nails are provided herein. The intramedullary nails can be used for the fixation of an epiphyseal fracture in a variety of long bones. For example, the long bone can be a femur, a fibula, a tibia, a humerus, a radius, or an ulna. The intramedullary nails can, in various aspects, be particularly useful for comminuted fractures involving the epiphysis of a long bone. These types of comminuted fractures are particularly difficult to treat using existing treatments that often require either partial prosthesis or external plating. In some embodiments, the intramedullary nails of the present disclosure can be used for the fixation of a radial head fracture, especially a Mason type II or Mason type III radial head fracture.

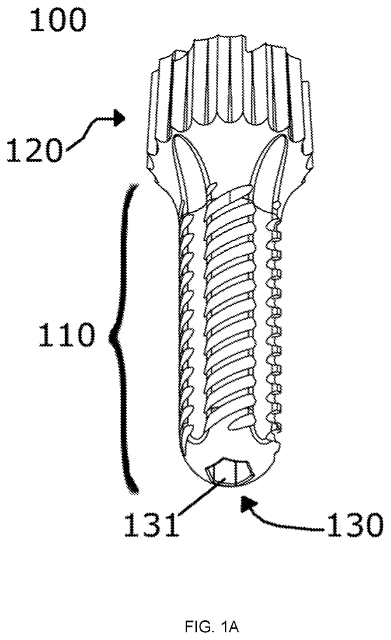

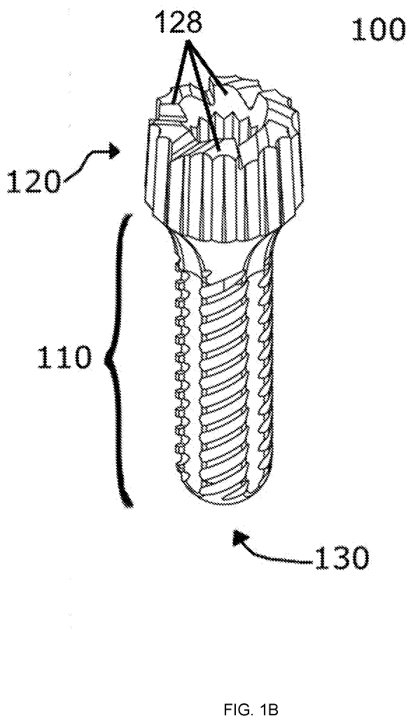

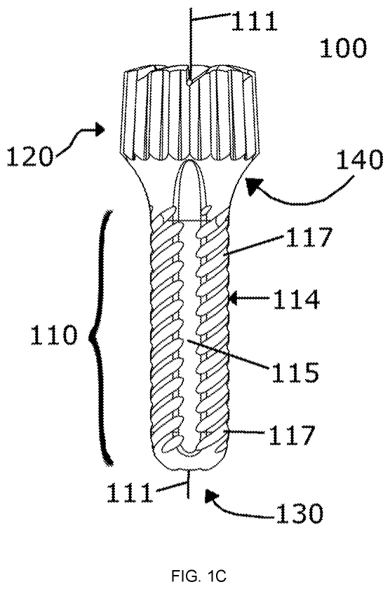

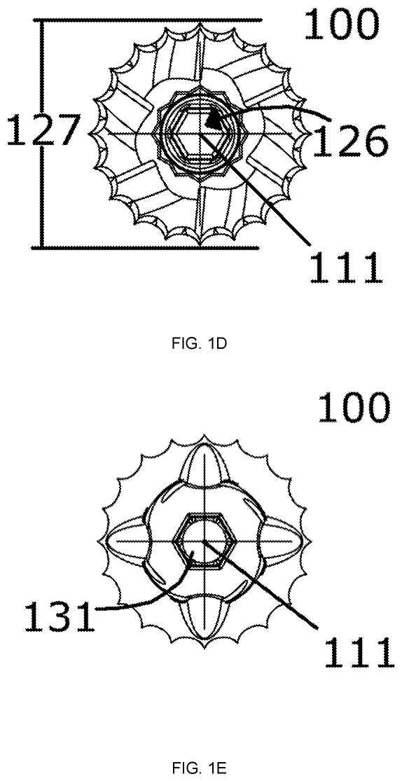

Exemplary embodiments of the intramedullary nail 100, 500, and 600 are depicted in FIGS. 1A-1E, FIGS. 2A-2D, FIGS. 3A-3B, FIGS. 4A-4F, FIGS. 5A-5F, and 6A-6F. In an exemplary embodiment, the intramedullary nail 100 has an intramedullary shaft portion 110 with a longer (longitudinal) axis 111 and a proximal end 112 and distal end 113. The intramedullary nail 100 has a cap portion 120 located at the proximal end 112 and a tip portion 130 located at the distal end 113. In some aspects, a tapered neck portion 140 connects the cap portion 120 to the proximal end 112 of the intramedullary shaft 110, although this need not necessarily be the case in all embodiments. The intramedullary nail can have an overall length (in the longitudinal direction) 101 of about 10 mm to about 500 mm, about 100 mm to 400 mm, about 200 mm to 300 mm, 15 mm to about 50 mm, about 20 mm to about 40 mm, about 25 mm to about 35 mm, or about 30 mm to 35 mm. The sizes and dimensions of the various aspects of the intramedullary nail can, in some aspects, be adjusted to accommodate the sizes and dimensions of the various long bones.

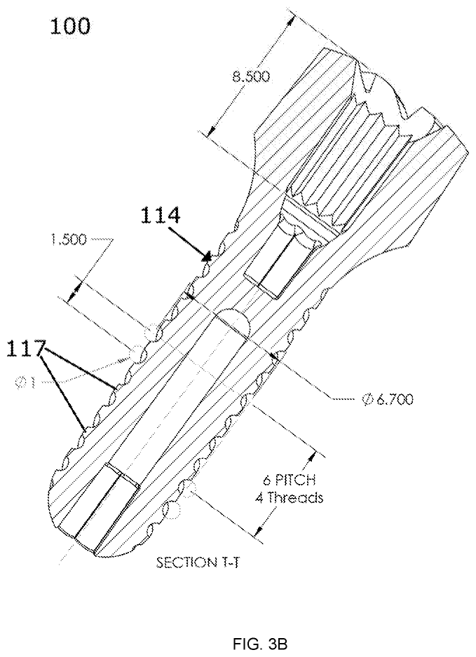

As depicted clearly in FIGS. 1A-1E, the cap portion 120 is located at the proximal end 112 of the intramedullary shaft 110. The cap portion 120 is configured for attaching thereto one or more epiphyseal bone fragments from the epiphyseal fracture. For example, in the exemplary embodiment depicted in FIGS. 1A-1E, the cap portion 120 has an outer surface 121 substantially parallel to the longitudinal axis 111 of the intramedullary shaft 110, and the outer surface 121 has a plurality of grooves 122 running longitudinally along the outer surface 121. The plurality of grooves 122 can be configured to receive one or more self-drilling screws for attaching the one or more epiphyseal bone fragments to the outer surface 121 of the cap portion 120. The grooves 122 on the outer surface 121 of the cap portion 120 can have a depth 123 as more clearly depicted in FIG. 3A. The depth 123 can be such that the grooves 122 aide in guiding the self-drilling screws for attaching the one or more epiphyseal bone fragments to the outer surface 121 of the cap portion 120. The grooves can have a depth 123 that can range from about 0.05 mm to about 2 mm. The grooves 122 can have a depth 123 of about 0.3 mm, about 0.4 mm, about 0.5 mm, about 0.6 mm and less than about 1.1 mm or less than about 0.9 mm. The plurality of grooves can include about 8 to 100, 10 to 50, about 10 to 30, or about 15 to 25 grooves. Although not necessarily the case in all embodiments, the grooves can be equally spaced circumferentially along the outer surface of the cap. The grooves can provide for easier placement of the self-drilling screws into the cap portion. Because the cap portion is configured to receive a self-drilling screw, the cap portion does not need to have pre-configured holes for attaching the epiphyseal bone fragments. This can allow for greater flexibility to handle a variety of epiphyseal bone fractures.

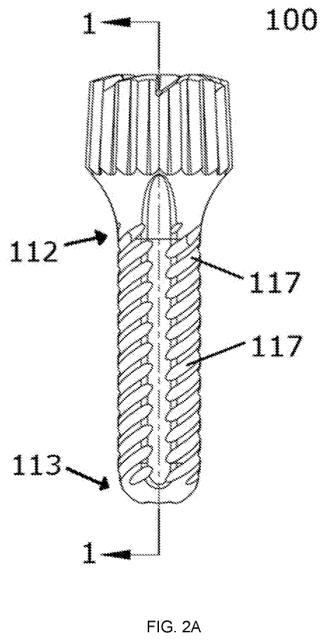

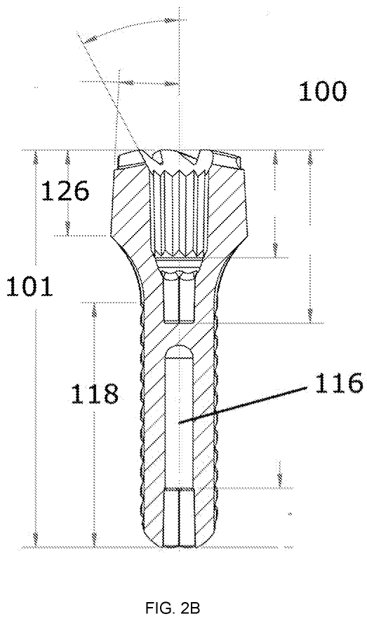

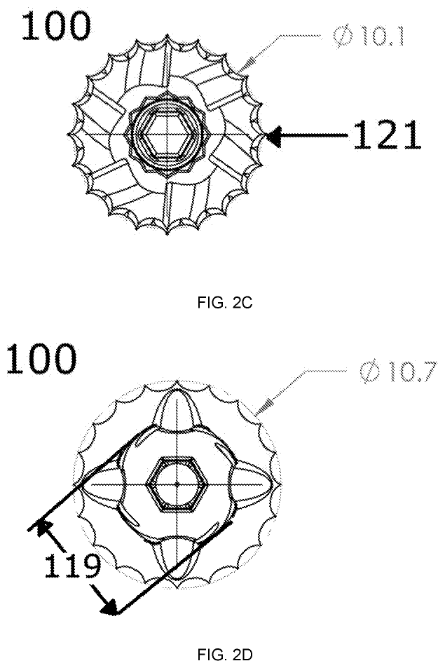

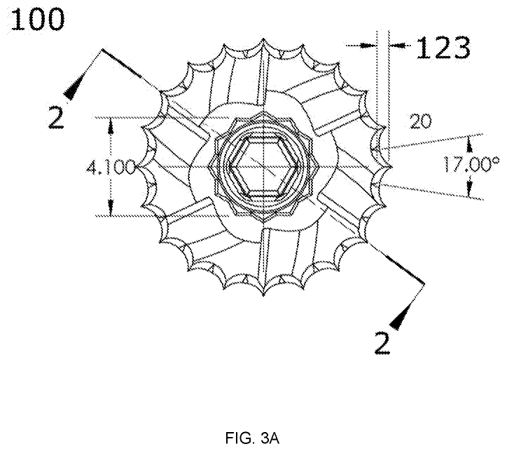

The cap portion 120 can have an upper surface 125 having an opening 126 dimensioned to receive a tip of a tool, for example for tightening the intramedullary nail 100 into the medullary canal and/or for securing the intramedullary nail during fixation of the epiphyseal bone fragments thereto. As more clearly depicted in FIG. 2B, the cap portion 120 can have a height 126 in the longitudinal direction that, in some aspects, can be about 5 mm to 30 mm, 5 mm to 15 mm, about 5 mm to 12 mm, about 5 mm to 10 mm, about 5 mm to 8 mm, or about 6.8 mm. The cap portion 120 can have an overall cylindrical shape, although it need not necessarily and in most embodiments will not be perfectly cylindrical. The cap portion 120 can have an outer diameter 127 of about 5 mm to about 30 mm, about 6 mm to 20 mm, about 6 mm to 15 mm, or about 8 mm to 12 mm. The cap portion 120 can have an outer diameter 127 that is larger than the outer diameter 119 of the intramedullary shaft 110. In some embodiments, the cap portion 120 has an opening 126 within the cap portion 120 with an internal diameter of about 2 mm to 8 mm, about 3 mm to 7 mm, or about 3 mm to 5 mm.

As shown at least in e.g. FIG. 1B the cap portion 120 can have at least 2 milling teeth 128 on the proximal surface of the cap portion 120. In some aspects, the cap portion 120 can have 6 milling teeth 128. The milling teeth 128 can help core the epiphysis of trabecular bone to create the cavity for the head of the screw to rest during implantation. The milling teeth 128 can have a depth that can range from about 0.5 mm to about 50 mm, about 10 mm to about 40 mm, or about 20 to about 30 mm. The contour of the milling teeth 128 can be tapered, beveled, squared, or any shape as desired. The milling teeth 128 can be blunt or sharp. In some aspects all the milling teeth 128 can be the same shape and/or size. In other aspects, the at least two of the milling teeth 128 are a different shape and/or size.

The intramedullary shaft 110 has a tip portion 130 located at its distal end 113 and configured for insertion of the intramedullary shaft 110 and the tip portion 130 into a medullary canal of the long bone. The intramedullary shaft 110 and the tip portion 130 will generally be shaped and dimensioned such that the intramedullary shaft 110 and the tip portion 130 can be inserted into the medullary canal securely and such that the cap portion 120 and the epiphyseal bone fragments attached thereto can be securely fixed in place to repair the epiphyseal fracture. The tip portion 130 can have an outer diameter that is smaller than the outer diameter 119 of the intramedullary shaft 110, e.g. the tip portion 130 can be tapered to facilitate ease of insertion into the medullary canal. The intramedullary shaft 110 can, in some embodiments, have an outer diameter 119 of about 5 mm to 30 mm, 3 mm to 10 mm, about 4 mm to 8 mm, about 5 mm to 7 mm, or about 6 mm. The intramedullary shaft 110 can have a length 118 (height in the longitudinal direction) of about 20 mm to 500 mm, 15 mm to 500 mm, 15 mm to 30 mm, about 15 mm to 25 mm, about 17 mm to 23 mm, or about 17 mm to 20 mm. The lengths, diameters, and other dimensions can be adjusted based upon the particular long bone and/or the particular patient to be treated.

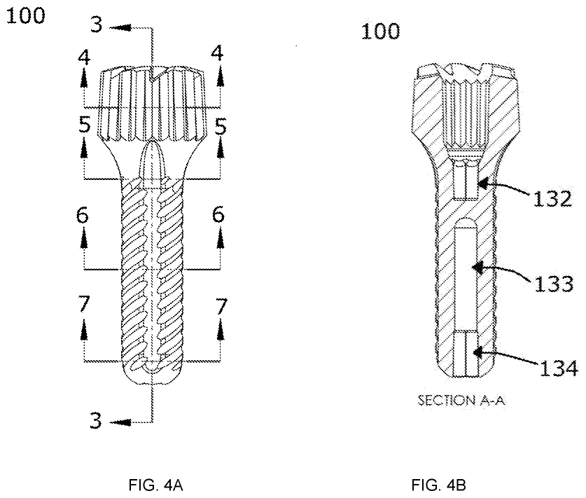

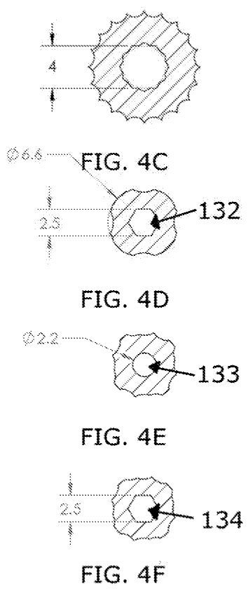

The tip portion 130 can have an opening 131 dimensioned to receive an end of a tool, for example for securing the intramedullary nail during fixation of the epiphyseal bone fragments thereto. In some aspects, the void 116 connects the opening 126 in the cap portion 120 to the opening 131 in the tip portion 130. This can be seen, for instance, in FIGS. 4A-4F. In this example, the void 116 has regions of differing shape and dimension in going from the opening 126 in the cap portion 120 to the opening 131 in the tip portion 130. For example, as depicted in FIG. 4D, the opening 126 in the cap portion 120 can include a first portion 132 that is configured hexagonally for receiving the end of a tool (see FIG. 4D), a second portion 133 that is cylindrically shaped (see FIG. 4E), and a third portion 134 at the opening 131 in the tip 130 that is hexagonally shaped (see FIG. 4F) for receiving the end of a tool.

The intramedullary shaft 110 can have an outer surface 114 substantially parallel to the longitudinal axis 111 and having a plurality of grooves 115 running longitudinally along the outer surface 114. The grooves 115 on the outer surface 114 of the intramedullary shaft 110 can have a depth such that the grooves 115 aide in guiding the self-drilling screws for securing the intramedullary shaft 110 within the medullary cavity. The grooves 115 can have a depth of about 0.3 mm, about 0.4 mm, about 0.5 mm, about 0.6 mm and less than about 1.5 mm, less than about 1.1 mm or less than about 0.9 mm. The plurality of grooves can include about 2 to 18, 2 to 8, 3 to 6, or about 5 grooves. Although not necessarily the case in all embodiments, the grooves can be equally spaced circumferentially along the outer surface 114 of the intramedullary shaft 110. The intramedullary shaft 110 can also include one or more void regions 116 sized and positioned to contain shavings from a self-drilling screw being inserted into the intramedullary shaft when the intramedullary nail is in place in the long bone. There can be one or a plurality of such void regions, e.g. in some aspects, there are one, two, three, or more internal void regions within the intramedullary shaft 110.

The intramedullary shaft 110 can have a plurality of threads 117 on the outer surface 114 that can, for instance, ease insertion of the intramedullary shaft 110 into the medullary canal and/or can provide easier fixation of the intramedullary shaft 110 within the medullary canal. The threads 117 can be male threads or female threads. They can have a thread form selected from a square thread form, a triangular thread form, a trapezoidal thread form, and a circular thread form among others. The threads can have any suitable pitch to needed to help secure the intramedullary nail in the intramedullary canal. The plurality of threads 117 can have a number of thread starts from about 1 to 10, about 2 to 8, about 2 to 6, or about 4. The height of the threads can be about 0.5 mm to about 3 mm, about 1 mm to about 2.5 mm, or about 1.5 to about 2 mm. The threads can have an outer diameter of about 0.5 mm to 5 mm, about 0.5 mm to 2.5 mm, or about 1 mm to 2 mm. The pitch, number of threads, and/or the dimensions of the threads can be adjusted to facilitate easier insertion and/or to improve the retainment of the intramedullary shaft 110 within the medullary canal.

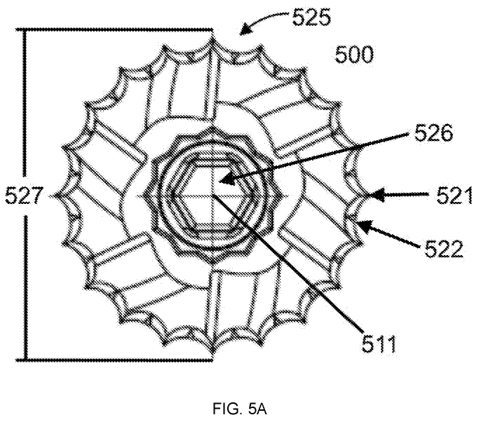

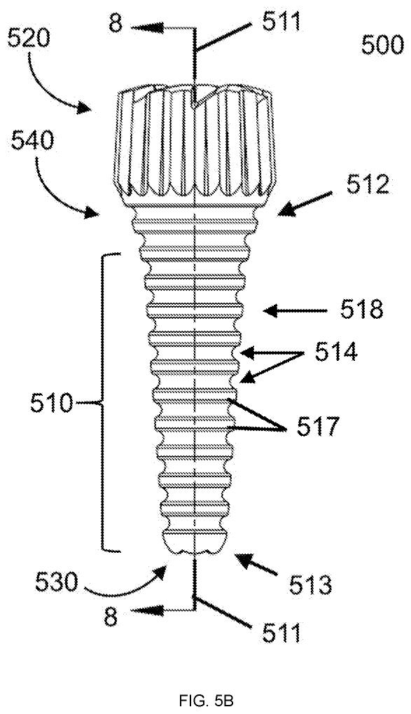

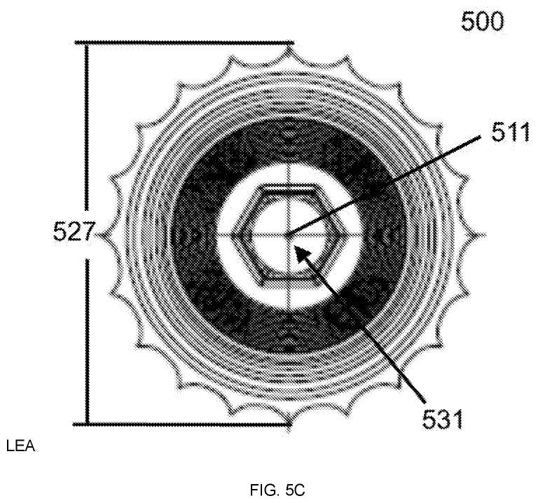

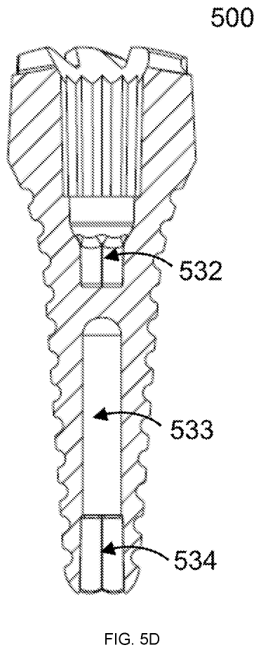

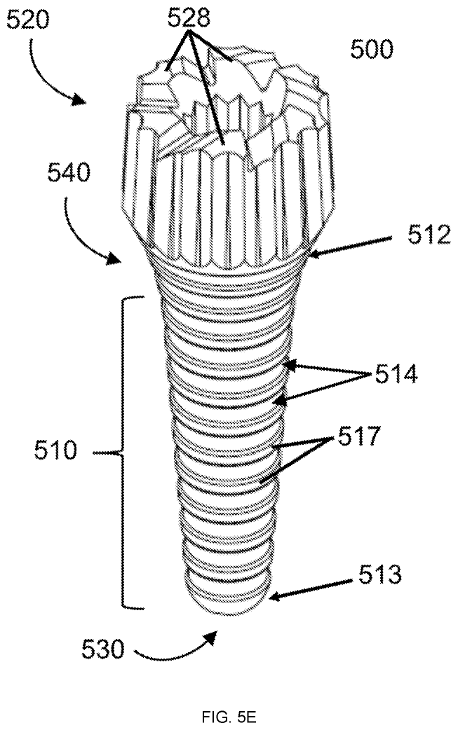

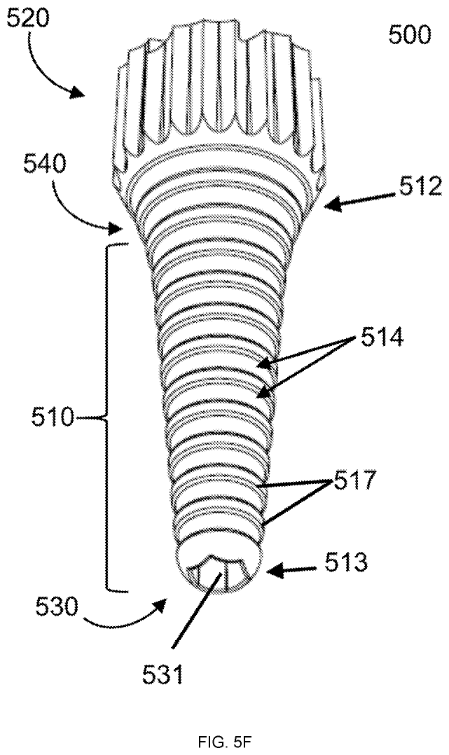

Discussion of another exemplary embodiment of the intramedullary nail continues with FIGS. 5A-5F, which depict an intramedullary nail 500 having a plurality of ridges 517 formed and a plurality of grooves 514 present on a shaft portion 510. The intramedullary nail 500 has an intramedullary shaft portion 510 with a longer (longitudinal) axis 511 and a proximal end 512 and distal end 513. The intramedullary nail 500 has a cap portion 520 located at the proximal end 512 and a tip portion 530 located at the distal end 513. In some aspects, a tapered neck portion 540 connects the cap portion 520 to the proximal end 512 of the intramedullary shaft 510, although this need not necessarily be the case in all embodiments. The intramedullary nail can have an overall length (in the longitudinal direction), as measured similarly to e.g. 101, FIG. 2B, of about 10 mm to about 500 mm, about 50 mm to 450 mm, about 100 mm to 400 mm, about 200 mm to about 300 mm, about 15 mm to about 50 mm, about 20 mm to about 40 mm, about 25 mm to about 35 mm, or about 30 mm to 35 mm. The sizes and dimensions of the various aspects of the intramedullary nail can, in some aspects, be adjusted to accommodate the sizes and dimensions of the various long bones.

As depicted clearly in FIGS. 5A-5F, the cap portion 520 is located at the proximal end 512 of the intramedullary shaft 510. The cap portion 520 is configured for attaching thereto one or more epiphyseal bone fragments from the epiphyseal fracture. For example, in the exemplary embodiment depicted in FIGS. 5A-5F, the cap portion 520 has an outer surface 521 substantially parallel to the longitudinal axis 511 of the intramedullary shaft 510, and the outer surface 521 has a plurality of grooves 522 running longitudinally along the outer surface 521. The plurality of grooves 522 can be configured to receive one or more self-drilling screws for attaching the one or more epiphyseal bone fragments to the outer surface 521 of the cap portion 520. The grooves 522 on the outer surface 521 of the cap portion 520 can have a depth (similar to e.g. 123, FIG. 3A). The depth can be such that the grooves 522 aide in guiding the self-drilling screws for attaching the one or more epiphyseal bone fragments to the outer surface 521 of the cap portion 520. The grooves can have a depth that can range from about 0.05 mm to about 2 mm. The grooves 522 can have a depth of about 0.3 mm, about 0.4 mm, about 0.5 mm, about 0.6 mm and less than about 1.1 mm or less than about 0.9 mm. The plurality of grooves can include about 8 to 100, 10 to 50, about 10 to 30, or about 15 to 25 grooves. Although not necessarily the case in all embodiments, the grooves can be equally spaced circumferentially along the outer surface of the cap.

The cap portion 520 can have an upper surface 525 having an opening 526 dimensioned to receive a tip of a tool, for example for tightening the intramedullary nail 500 into the medullary canal and/or for securing the intramedullary nail during fixation of the epiphyseal bone fragments thereto. The cap portion 520 can have a height (similar to 126, FIG. 2B) in the longitudinal direction that, in some aspects, can be about 5 mm to 30 mm, 5 mm to 15 mm, about 5 mm to 12 mm, about 5 mm to 10 mm, about 5 mm to 8 mm, or about 6.8 mm. The cap portion 520 can have an overall cylindrical shape, although it need not necessarily and in most embodiments will not be perfectly cylindrical. The cap portion 520 can have an outer diameter 527 of about 5 mm to 30 mm, about 6 mm to 20 mm, about 6 mm to 15 mm, or about 8 mm to 12 mm. In some embodiments, the cap portion 120 has an opening 126 within the cap portion 120 with an internal diameter of about 2 mm to 8 mm, about 3 mm to 7 mm, or about 3 mm to 5 mm.

As shown at least in e.g. FIG. 5E the cap portion 520 can have at least 2 milling teeth 528 on the proximal surface of the cap portion 520. In some aspects, the cap portion 520 can have 6 milling teeth 528. The milling teeth 528 can help core the epiphysis of trabecular bone to create the cavity for the head of the screw to rest during implantation. The milling teeth 528 can have a depth that can range from about 0.5 mm to about 50 mm, about 10 mm to about 40 mm, or about 20 to about 30 mm. The contour of the milling teeth 528 can be tapered, beveled, squared, or any shape as desired. The milling teeth 528 can be blunt or sharp. In some aspects all the milling teeth 528 can be the same shape and/or size. In other aspects, the at least two milling teeth 528 are a different shape and/or size.

The intramedullary shaft 510 has a tip portion 530 located at its distal end 513 and configured for insertion of the intramedullary shaft 510 and the tip portion 530 into a medullary canal of the long bone. The intramedullary shaft 510 and the tip portion 530 will generally be shaped and dimensioned such that the intramedullary shaft 510 and the tip portion 530 can be inserted into the medullary canal securely and such that the cap portion 520 and the epiphyseal bone fragments attached thereto can be securely fixed in place to repair the epiphyseal fracture. The tip portion 530 can have an outer diameter that is smaller than the outer diameter (see e.g. 119, FIG. 2D) of the intramedullary shaft 510, e.g. the tip portion 530 can be tapered to facilitate ease of insertion into the medullary canal. The intramedullary shaft 510 can, in some embodiments, have an outer diameter of about 5 mm to 30 mm, 3 mm to 10 mm, about 4 mm to 8 mm, about 5 mm to 7 mm, or about 6 mm. The intramedullary shaft 510 can have a length (see e.g. 118, FIG. 2B) (height in the longitudinal direction) of about 20 mm to 500 mm, 15 mm to 500 mm, 15 mm to 30 mm, about 15 mm to 25 mm, about 17 mm to 23 mm, or about 17 mm to 20 mm. The lengths, diameters, and other dimensions can be adjusted based upon the particular long bone and/or the particular patient to be treated.

The tip portion 530 can have an opening 531 dimensioned to receive an end of a tool, for example for securing the intramedullary nail during fixation of the epiphyseal bone fragments thereto. In some aspects, the void (similar to 116, FIGS. 4A-4F) connects the opening 526 in the cap portion 520 to the opening 531 in the tip portion 530. This can be seen, for instance, in FIG. 5D. The void can have regions of differing shape and dimension in going from the opening 526 in the cap portion 520 to the opening 531 in the tip portion 530. For example, as depicted in FIG. 5D, the opening 526 in the cap portion 520 can include a first portion 532 that is configured hexagonally for receiving the end of a tool (see FIG. 5A), a second portion 533 that is cylindrically shaped (similar to that shown in e.g. FIG. 4E), and a third portion 534 at the opening 531 in the tip 530 that is hexagonally shaped (see FIG. 5C) for receiving the end of a tool.

The intramedullary shaft 510 can have an outer surface 518 substantially parallel to the longitudinal axis 511 and having a plurality of grooves 514 and ridges 517 running substantially perpendicular to the longitudinal axis 511 along the outer surface 518. The plurality of grooves 514 can include about 2 to 50, 10 to 40, 20-30, grooves 514. The plurality of ridges 517 can include about 2 to 50, 10 to 40, 20-30, ridges 517. Although not necessarily the case in all embodiments, the grooves 514 and/or ridges 517 can be equally spaced circumferentially along the outer surface 518 of the intramedullary shaft 510. The grooves 514 can have any concave contour, including but not limited to, rounded, square, angled, and irregular. The grooves 514 can have a depth at their deepest point, as measured from the highest portion of a neighboring ridge 517, that can range from about 0.5 mm to about 3 mm, about 1 mm to about 2.5 mm, or about 1.5 to about 2 mm. The ridges 517 can have a height as measured from the deepest point of a neighboring groove 514, that can range from about 0.5 mm to about 3 mm, about 1 mm to about 2.5 mm, or about 1.5 to about 2 mm. The ridges 517 can have any convex contour, including but not limited to, rounded, square, angled, and irregular. The numbers of and dimensions of the ridges 517 and/or grooves 514 can be adjusted to facilitate easier insertion and/or to improve the retention of the intramedullary shaft 110 within the medullary canal.

The intramedullary shaft 510 can also include one or more void regions sized and positioned to contain shavings from a self-drilling screw being inserted into the intramedullary shaft when the intramedullary nail is in place in the long bone. There can be one or a plurality of such void regions, e.g. in some aspects, there are one, two, three, or more internal void regions within the intramedullary shaft 510.

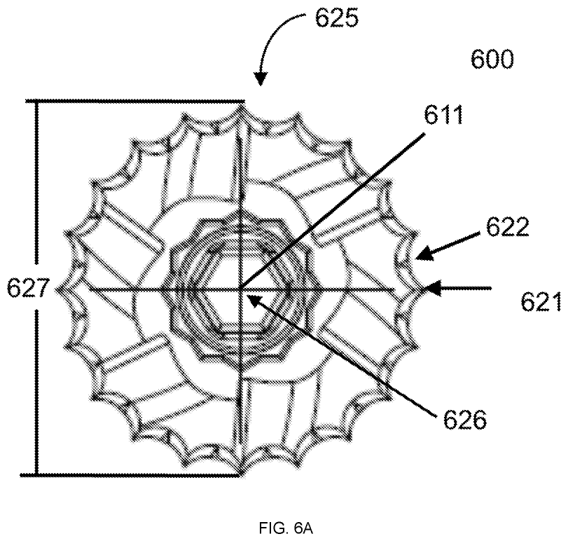

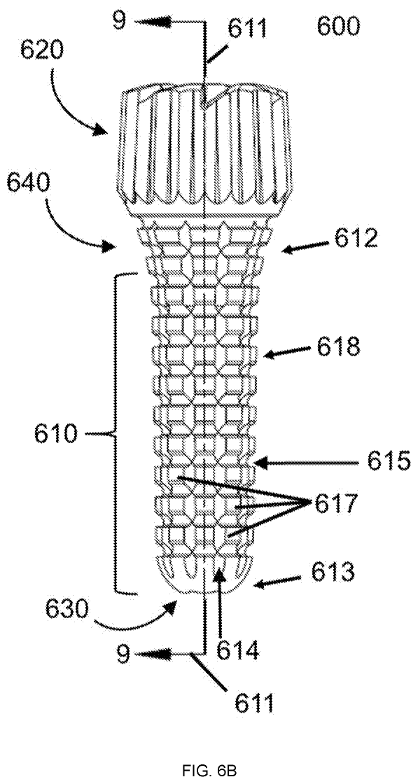

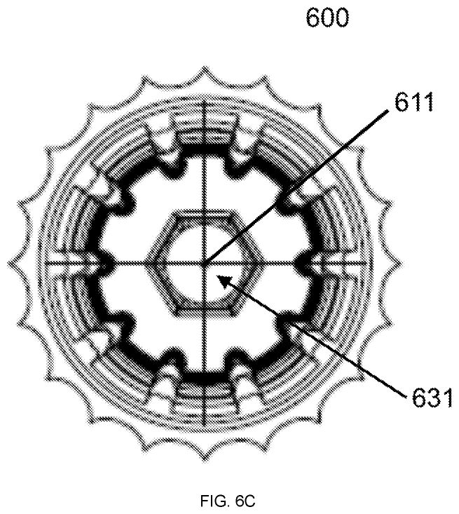

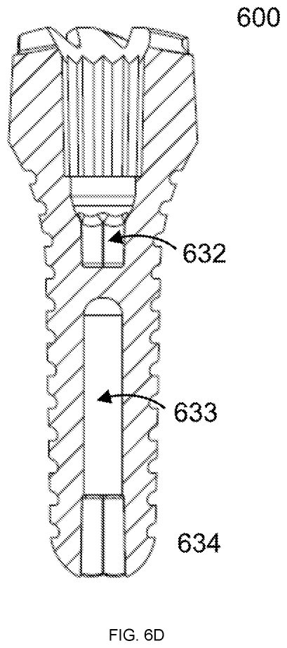

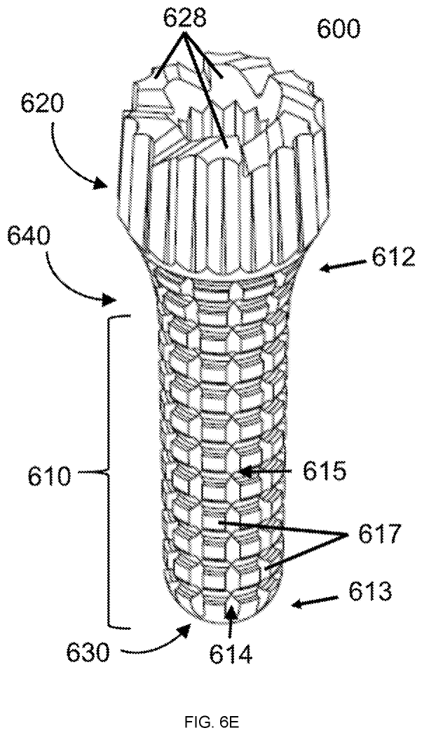

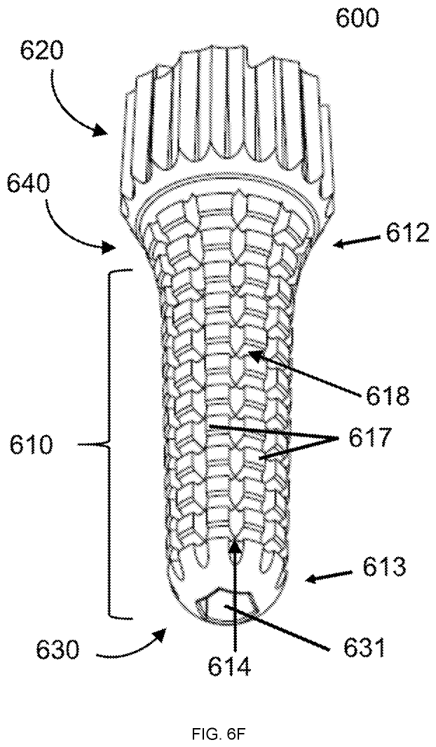

Discussion of another exemplary embodiment of the intramedullary nail continues with FIGS. 6A-6F, which depict an intramedullary nail 600 having a broached surface created by a plurality of raised portions 617 that can be positioned on an outer surface 618 of a intramedullary shaft portion 610 that forms longitudinal grooves 614 that can run substantially parallel to the longitudinal axis 611 and perpendicular grooves 615 that can run substantially perpendicular to the longitudinal axis 611 on the outer surface 618. The intramedullary nail 600 has an intramedullary shaft portion 610 with a longer (longitudinal) axis 611 and a proximal end 612 and distal end 613. The intramedullary nail 600 has a cap portion 620 located at the proximal end 612 and a tip portion 630 located at the distal end 613. In some aspects, a tapered neck portion 640 connects the cap portion 620 to the proximal end 612 of the intramedullary shaft 610, although this need not necessarily be the case in all embodiments. The intramedullary nail 600 can have an overall length (in the longitudinal direction), as measured similarly to e.g. 101, FIG. 2B, of about 10 mm to about 500 mm, about 50 mm to 450 mm, about 100 mm to 400 mm, about 200 mm to about 300 mm, about 15 mm to about 50 mm, about 20 mm to about 40 mm, about 25 mm to about 35 mm, or about 30 mm to 35 mm. The sizes and dimensions of the various aspects of the intramedullary nail can, in some aspects, be adjusted to accommodate the sizes and dimensions of the various long bones.

As depicted clearly in FIGS. 6A-6F, the cap portion 620 is located at the proximal end 612 of the intramedullary shaft 610. The cap portion 620 is configured for attaching thereto one or more epiphyseal bone fragments from the epiphyseal fracture. For example, in the exemplary embodiment depicted in FIGS. 6A-6F, the cap portion 620 has an outer surface 621 substantially parallel to the longitudinal axis 611 of the intramedullary shaft 610, and the outer surface 621 has a plurality of grooves 622 running longitudinally along the outer surface 621. The plurality of grooves 622 can be configured to receive one or more self-drilling screws for attaching the one or more epiphyseal bone fragments to the outer surface 621 of the cap portion 620. The grooves 622 on the outer surface 621 of the cap portion 620 can have a depth (similar to e.g. 123, FIG. 3A). The depth can be such that the grooves 622 aide in guiding the self-drilling screws for attaching the one or more epiphyseal bone fragments to the outer surface 621 of the cap portion 620. The grooves can have a depth that can range from about 0.05 mm to about 2 mm. The grooves 622 can have a depth of about 0.3 mm, about 0.4 mm, about 0.5 mm, about 0.6 mm and less than about 1.1 mm or less than about 0.9 mm. The plurality of grooves can include about 8 to 100, 10 to 50, about 10 to 30, or about 15 to 25 grooves. Although not necessarily the case in all embodiments, the grooves can be equally spaced circumferentially along the outer surface of the cap.

The cap portion 620 can have an upper surface 625 having an opening 626 dimensioned to receive a tip of a tool, for example for tightening the intramedullary nail 600 into the medullary canal and/or for securing the intramedullary nail during fixation of the epiphyseal bone fragments thereto. The cap portion 620 can have a height (similar to 126, FIG. 2B) in the longitudinal direction that, in some aspects, can be about 5 mm to 30 mm, 5 mm to 15 mm, about 5 mm to 12 mm, about 5 mm to 10 mm, about 5 mm to 8 mm, or about 6.8 mm. The cap portion 620 can have an overall cylindrical shape, although it need not necessarily and in most embodiments will not be perfectly cylindrical. The cap portion 620 can have an outer diameter 627 of about 5 mm to 30 mm, about 6 mm to 20 mm, about 6 mm to 15 mm, or about 8 mm to 12 mm. In some embodiments, the cap portion 620 has an opening 626 within the cap portion 620 with an internal diameter of about 2 mm to 8 mm, about 3 mm to 7 mm, or about 3 mm to 5 mm.

As shown at least in e.g. FIG. 6E the cap portion 620 can have at least 2 milling teeth 628 on the proximal surface of the cap portion 620. In some aspects, the cap portion 620 can have 6 milling teeth 628. The milling teeth 628 can help core the epiphysis of trabecular bone to create the cavity for the head of the screw to rest during implantation. The milling teeth 628 can have a depth that can range from about 0.5 mm to about 50 mm, about 10 mm to about 40 mm, or about 20 to about 30 mm. The contour of the milling teeth 628 can be tapered, beveled, squared, or any shape as desired. The milling teeth 628 can be blunt or sharp. In some aspects all the milling teeth 628 can be the same shape and/or size. In other aspects, the at least two of the milling teeth 628 are a different shape and/or size.

The intramedullary shaft 610 has a tip portion 630 located at its distal end 613 and configured for insertion of the intramedullary shaft 610 and the tip portion 630 into a medullary canal of the long bone. The intramedullary shaft 610 and the tip portion 630 will generally be shaped and dimensioned such that the intramedullary shaft 610 and the tip portion 630 can be inserted into the medullary canal securely and such that the cap portion 620 and the epiphyseal bone fragments attached thereto can be securely fixed in place to repair the epiphyseal fracture. The tip portion 630 can have an outer diameter that is smaller than the outer diameter (see e.g. 119, FIG. 2D) of the intramedullary shaft 610, e.g. the tip portion 630 can be tapered to facilitate ease of insertion into the medullary canal. The intramedullary shaft 610 can, in some embodiments, have an outer diameter of about 5 mm to 30 mm, 3 mm to 10 mm, about 4 mm to 8 mm, about 5 mm to 7 mm, or about 6 mm. The intramedullary shaft 610 can have a length (see e.g. 118, FIG. 2B) (height in the longitudinal direction) of about 20 mm to 500 mm, 15 mm to 500 mm, 15 mm to 30 mm, about 15 mm to 25 mm, about 17 mm to 23 mm, or about 17 mm to 20 mm. The lengths, diameters, and other dimensions can be adjusted based upon the particular long bone and/or the particular patient to be treated.

The tip portion 630 can have an opening 631 dimensioned to receive an end of a tool, for example for securing the intramedullary nail during fixation of the epiphyseal bone fragments thereto. In some aspects, the void (similar to 116, FIGS. 4A-4F) connects the opening 626 in the cap portion 620 to the opening 631 in the tip portion 630. This can be seen, for instance, in FIG. 6D. The void can have regions of differing shape and dimension in going from the opening 626 in the cap portion 620 to the opening 631 in the tip portion 630. For example, as depicted in FIG. 6D, the opening 626 in the cap portion 620 can include a first portion 632 that is configured hexagonally for receiving the end of a tool (see FIG. 6A), a second portion 633 that is cylindrically shaped (similar to that shown in e.g. FIG. 4E), and a third portion 634 at the opening 631 in the tip 630 that is hexagonally shaped (see FIG. 6C) for receiving the end of a tool.

The intramedullary shaft 610 can have an outer surface 618 substantially parallel to the longitudinal axis 611. The outer surface 618 can have a plurality of raised portions 617 that can be positioned on an outer surface 618 of a intramedullary shaft portion 610 that forms longitudinal grooves 614 that can run substantially parallel to the longitudinal axis 611 and perpendicular grooves 615 that can run substantially perpendicular to the longitudinal axis 611 on the outer surface 618. The raised portions 617 can be any size and can be any regular shape (square, rectangular, triangular, round, hexagonal, etc.) or any irregular shape. The raised portions can have a base region, which is closest to the center of the intramedullary nail and a surface portion, which forms the outermost part of the outer surface 618. The raised portions 617 can be optionally tapered along their height such that the base of a raised portion can be larger than the surface of the raised portion. The base of the raised portions 617 can, in any one dimension (length, width, radius, etc.), range from about 0.01 mm to about 10 mm, about 0.5 to about 9.5 mm, about 1 mm to about 9 mm, about 2 mm to about 8 mm, about 3 mm to about 7 mm, about 4 mm to about 6 mm, or about 5 mm. The surface of the raised portions 617 can, in any one dimension (length, width, radius, etc.), range from about 0.01 mm to about 10 mm, about 0.5 to about 9.5 mm, about 1 mm to about 9 mm, about 2 mm to about 8 mm, about 3 mm to about 7 mm, about 4 mm to about 6 mm, or about 5 mm. Optionally, the raised portions 617 can all be substantially the same size and/or shape. In other aspects, at least two raised portions 617 can be different in size and/or shape. The outer surface 618 can optionally be configured such that the raised portions 617 are equally longitudinally and/or equilaterally spaced from one another. In other aspects, the outer surface 618 can be configured such that the raised portions 617 are not longitudinally and/or equilaterally spaced from one another. The number of raised portions 617 can range from about 10 to about 1000 or more, 100 to about 900 about 200 to about 800, about 300 to about 700, about 400 to about 600, or about 500.

The raised portions 617 can have a height, as measured from the deepest point of a neighboring groove 614, 615 that can range from about 0.5 mm to about 3 mm, about 1 mm to about 2.5 mm, or about 1.5 to about 2 mm. The height of all the raised portions 617 can be the same or in some aspects, at least two of the raised portions 617 can have a different height. The longitudinal grooves 614 can have a depth, as measured from the surface of a neighboring raised portion 617, that can range from about 0.5 mm to about 3 mm, about 1 mm to about 2.5 mm, or about 1.5 to about 2 mm. The depth of all the longitudinal grooves 614 can be the same or in some aspects, at least two of the longitudinal grooves 614 can have a different depth. The perpendicular grooves 615 can have a depth, as measured from the surface of a neighboring raised portion 617, that can range from about 0.5 mm to about 3 mm, about 1 mm to about 2.5 mm, or about 1.5 to about 2 mm. The depth of all the perpendicular grooves 615 can be the same or in some aspects, at least two of the perpendicular grooves 615 can have a different depth. The grooves 614, 615 can have any concave contour, including but not limited to, rounded, square, angled, and irregular. The numbers of and dimensions of the raised portions 617 and/or grooves 614, 615 can be adjusted to facilitate easier insertion and/or to improve the retainment of the intramedullary shaft 610 within the medullary canal.

The intramedullary shaft 610 can also include one or more void regions sized and positioned to contain shavings from a self-drilling screw being inserted into the intramedullary shaft when the intramedullary nail is in place in the long bone. There can be one or a plurality of such void regions, e.g. in some aspects, there are one, two, three, or more internal void regions within the intramedullary shaft 610.

A variety of materials can be used to make the intramedullary nails of the instance disclosure, and the material properties can be chosen to enhance the bone regrowth and/or to prevent unwanted side effects such as infection. In some aspects, the intramedullary nail is made partially or entirely from a thermoplastic polymer. Thermoplastic polymers can include one or more polymers selected from a polyethylene, a polymethylmethacrylate, a polyurethane, a polysulfone, a polyetherimide, a polyimide, an ultra-high molecular weight polyethylene (UHMWPE), a cross-linked UHMWPE, a polyaryletherketone, a copolymer thereof, or a blend thereof. In some embodiments, the thermoplastic polymer will be entirely or partially or will include a polyaryletherketone. Suitable polyaryletherketones can include poly(aryl-ether-ether-ketone) (PEEK), polyetherketoneketone (PEKK), and poly(aryl-ether-ketone-ether-ketoneketone) (PEKEKK). PEEK is particularly suitable because its modulus of elasticity closely matches that of bone. However, PEEK is also a hydrophobic material and bacteria tend to adhere easily to these types of surfaces. In some embodiments a thermoplastic resin material, such as PEEK, is modified to increase surface hydrophobicity and/or is coated with an antibacterial agent. The materials can be further modified, e.g. to increase surface hydrophobicity and/or by adding antibacterial agents to the surface.

Uses of Intramedullary Nails for Fixation of Epiphyseal Fractures

The intramedullary nails of the present disclosure can be used in a variety of methods for fixation of an epiphyseal fracture in a long bone of a patient in need thereof. The patient can include a human patient or a non-human patient, e.g. a land mammal such as a dog, a cat, a horse, a cow, a sheep, or a goat. In some embodiments, the patient is a human patient, the long bone is a radius, and the epiphyseal fracture is a radial head fracture.

The intramedullary nails of the present disclosure can be used for fixation of an epiphyseal fracture in a variety of long bones. The long bone can include a femur, a fibula, a tibia, a humerus, a radius, or an ulna. The intramedullary nails are particularly well suited for fixation of epiphyseal fracture is comminuted, e.g. wherein the epiphyseal bone fragments include 2, 3, 4, or more bone fragments. In such instances, the epiphyseal bone fragments can be attached to the cap portion of the intramedullary nail to recreate the head of the long bone, which can then be attached to the diphysis by securing the intramedullary shaft into the medullary canal of the long bone. This can provide for improved fixation of the epiphyseal fracture without the need for bulky external hardware that can hinder joint function and cause irritation. The epiphyseal fracture can include a Salter-Harris type 1, type 2, type 3, or type 4 fracture with comminution. As used herein, the term “comminution” refers to fractures where the bone breaks into multiple pieces. A comminuted epiphyseal fracture is particularly difficult to treat with conventional methods, because the epiphysis breaks into multiple fragments that typically require an external form of fixation or a prosthesis.

Methods for the fixation of an epiphyseal fracture in a long bone of a patient in need thereof are provided. The methods can include attaching one or more epiphyseal bone fragments to a cap portion on a proximal end of an intramedullary nail of the instant disclosure, and inserting a distal end of the intramedullary nail into a medullary canal of the long bone. The methods can include fixation of the intramedullary shaft of the nail in the medullary canal, either using mechanical fixation such as a small self-drilling screw or using a bone cement such as polymethyl methacrylate (PMMA). The methods can further include inserting one or more self-drilling screws through an outer surface of the diaphysis and into the intramedullary shaft of the intramedullary nail to secure the intramedullary nail having the one or more epiphyseal bone fragments attached thereto. The methods can also include applying a bone cement such as PMMA to the intramedullary shaft prior to insertion into the medullary canal.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

The present disclosure will be better understood upon review of the following features, which should not be confused with the claims.

Feature 1. An intramedullary nail for fixation of an epiphyseal fracture in a long bone of a patient in need thereof, the intramedullary nail comprising: an intramedullary shaft configured for insertion into a medullary canal of the long bone, the intramedullary shaft having a longitudinal axis, a proximal end, and a distal end; a cap portion at the proximal end and configured for attaching thereto one or more epiphyseal bone fragments from the epiphyseal fracture; and a tip portion at the distal end and configured for insertion into the medullary canal of the long bone.

Feature 2. The intramedullary nail according to feature 1, wherein the intramedullary shaft is configured to be inserted into the medullary canal of the long bone from an end of the long bone.

Feature 3. The intramedullary nail according to any one of features 1-2, wherein the intramedullary shaft is configured to be inserted into the medullary canal of the long bone from an end of the long bone having the epiphyseal fracture such that, when inserted into the medullary canal, the intramedullary shaft is approximately parallel to a long axis of the long bone.

Feature 4. The intramedullary nail according to any one of features 1-3, wherein the intramedullary shaft is configured to be inserted into the medullary canal of the long bone from an end of the long bone having the epiphyseal fracture such that, when inserted into the medullary canal, the intramedullary shaft is inserted along a length of the medullary canal.

Feature 5. The intramedullary nail according to any one of features 1-4, wherein the intramedullary shaft is configured such that, when the intramedullary shaft is inserted along the length of the medullary canal, the intramedullary shaft secures the cap portion at the end of the long bone having the epiphyseal fracture.

Feature 6. The intramedullary nail according to any one of features 1-5, wherein the cap portion is configured such that, when the one or more epiphyseal bone fragments are attached to the cap portion and the cap portion is secured at the end of the long bone having the epiphyseal fracture, the intramedullary nail provides for fixation of the epiphyseal fracture.

Feature 7. The intramedullary nail according to any one of features 1-6, wherein the intramedullary shaft comprises an outer surface substantially parallel to the longitudinal axis of the intramedullary shaft, and wherein the outer surface comprises a plurality of grooves running longitudinally along the outer surface.

Feature 8. The intramedullary nail according to any one of features 1-7, wherein the plurality of grooves on the outer surface of the intramedullary shaft comprise about 2 to 8 grooves equally spaced circumferentially along the outer surface of the intramedullary shaft.

Feature 9 The intramedullary nail according to any one of features 1-8, wherein the grooves on the outer surface of the intramedullary shaft have a depth of about 0.3 mm to about 1.5 mm.

Feature 10. The intramedullary nail according to any one of features 1-9, wherein the intramedullary shaft has an outer surface having a plurality of threads on the outer surface.

Feature 11. The intramedullary nail according to any one of features 1-10, wherein the threads are female threads.

Feature 12. The intramedullary nail according to any one of features 1-11, wherein the threads are male threads.

Feature 13. The intramedullary nail according to any one of features 1-12, wherein the threads have a thread form selected from the group consisting of a square thread form, a triangular thread form, a trapezoidal thread form, and a circular thread form.

Feature 14. The intramedullary nail according to any one of features 1-13, wherein the plurality of threads have a number of thread starts from about 2 to 8.

Feature 15. The intramedullary nail according to any one of features 1-14, wherein each of the threads in the plurality of threads have an outer diameter of about 1 mm to about 2 mm.

Feature 16. The intramedullary nail according to any one of features 1-15, wherein each of the threads in the plurality of threads have a height of about 0.5 mm to about 3 mm.

Feature 17. The intramedullary nail according to any one of features 1-16, wherein the intramedullary shaft comprises an outer surface substantially parallel to the longitudinal axis of the intramedullary shaft, and wherein the outer surface comprises a broached surface.

Feature 18. The intramedullary nail according to any one of features 1-17, wherein the intramedullary shaft has a height in the longitudinal direction of about 15 mm to about 25 mm.

Feature 19. The intramedullary nail according to any one of features 1-18, wherein the intramedullary shaft has an outer diameter of about 4 mm to about 8 mm.

Feature 20. The intramedullary nail according to any one of features 1-19, wherein the intramedullary nail is made partially or entirely from a thermoplastic polymer.

Feature 21. The intramedullary nail according to any one of features 1-20, wherein the thermoplastic polymer comprises one or more polymers selected from the group consisting of a polyethylene, a polymethylmethacrylate, a polyurethane, a polysulfone, a polyetherimide, a polyimide, an ultra-high molecular weight polyethylene (UHMWPE), a cross-linked UHMWPE, a polyaryletherketone, a copolymer thereof, and a blend thereof.

Feature 22. The intramedullary nail according to any one of features 1-21, wherein the thermoplastic polymer comprises a polyaryletherketone.

Feature 23. The intramedullary nail according to any one of features 1-22, wherein the polyaryletherketone is selected from the group consisting of poly(aryl-ether-ether-ketone) (PEEK), polyetherketoneketone (PEKK), and poly(aryl-ether-ketone-ether-ketoneketone) (PEKEKK).

Feature 24. The intramedullary nail according to any one of features 1-23, wherein the thermoplastic polymer has been modified to increase surface hydrophobicity.

Feature 25. The intramedullary nail according to any one of features 1-24, further comprising an antibacterial agent on one or more surfaces.

Feature 26. The intramedullary nail according to any one of features 1-25, wherein the cap portion is configured to receive one or more self-drilling screws for attaching the one or more epiphyseal bone fragments to the cap portion.

Feature 27. The intramedullary nail according to any one of features 1-26, wherein the cap portion comprises an outer surface substantially parallel to the longitudinal axis of the intramedullary shaft, and wherein the outer surface comprises a plurality of grooves running longitudinally along the outer surface.

Feature 28. The intramedullary nail according to any one of features 1-27, wherein the plurality of grooves on the outer surface of the cap are configured to receive one or more self-drilling screws for attaching the one or more epiphyseal bone fragments to the outer surface of the cap portion.

Feature 29. The intramedullary nail according to any one of features 1-28, wherein the grooves on the outer surface of the cap have a depth of about 0.3 mm to about 0.9 mm.

Feature 30. The intramedullary nail according to any one of features 1-29, wherein the plurality of grooves on the outer surface of the cap comprise about 15 to 25 grooves equally spaced circumferentially along the outer surface of the cap.

Feature 31. The intramedullary nail according to any one of features 1-30, wherein the cap portion further comprise an upper surface having an opening dimensioned to receive a tip of a tool for tightening the intramedullary nail into the medullary canal.

Feature 32. The intramedullary nail according to any one of features 1-31, wherein the cap portion has a height in the longitudinal direction of about 6 mm to about 10 mm.

Feature 33. The intramedullary nail according to any one of features 1-32, wherein the cap portion has an outer diameter of about 6 mm to about 15 mm

Feature 34. The intramedullary nail according to any one of features 1-33, wherein the intramedullary shaft comprises one or more void regions sized and positioned to contain one or more shavings from a self-drilling screw being inserted into the intramedullary shaft.

Feature 35. The intramedullary nail according to any one of features 1-34, wherein the intramedullary shaft comprises about 1 to 3 void regions located along the longitudinal axis of the intramedullary shaft.

Feature 36. The intramedullary nail according to any one of features 1-35, wherein the patient is a human patient.

Feature 37. The intramedullary nail according to any one of features 1-36, wherein the long bone is selected from the group consisting of a femur, a fibula, a tibia, a humerus, a radius, and an ulna.

Feature 38. The intramedullary nail according to any one of features 1-37, wherein the patient is a land mammal.

Feature 39. The intramedullary nail according to any one of features 1-38, wherein the patient is selected from the group consisting of a dog, a cat, a horse, a cow, a sheep, and a goat.

Feature 40. The intramedullary nail according to any one of features 1-39, wherein the patient is a human patient and the long bone is a radius.

Feature 41. A method of fixation of an epiphyseal fracture in a long bone of a patient in need thereof, the method comprising: attaching one or more epiphyseal bone fragments from the epiphyseal fracture to a cap portion on a proximal end of an intramedullary nail, and inserting a distal end of the intramedullary nail into a medullary canal of the long bone.

Feature 42. A method of fixation of an epiphyseal fracture in a long bone of a patient in need thereof, the method comprising: attaching one or more epiphyseal bone fragments from the epiphyseal fracture to the cap portion of an intramedullary nail according to any one of features 1-40, and inserting the distal end of the intramedullary nail into a medullary canal of the long bone.

Feature 43. A method of fixation of an epiphyseal fracture in a long bone of a patient in need thereof, the method comprising: attaching one or more epiphyseal bone fragments from the epiphyseal fracture to an intramedullary nail, wherein the intramedullary nail comprises (i) an intramedullary shaft having a longitudinal axis, a proximal end, and a distal end, (ii) a cap portion at the proximal end, and (iii) a tip portion at the distal end, wherein the one or more epiphyseal bone fragments are attached to the cap portion; and inserting the distal end of the intramedullary shaft into the medullary canal at an end of the long bone having the epiphyseal fracture such that the intramedullary shaft is secured along a length of the medullary canal thereby securing the epiphyseal bone fragments at an epiphyseal region of the long bone.

Feature 44. The method according to any one of features 41-43, wherein the inserting step comprises inserting the tip portion of the intramedullary shaft into the medullary canal at an end of the long bone having the epiphyseal fracture.

Feature 45. The method according to any one of features 41-44, wherein the inserting step comprises inserting the intramedullary shaft into the medullary canal such that the intramedullary shaft is approximately parallel to a long axis of the long bone.

Feature 46. The method according to any one of features 41-45, wherein the inserting step comprises inserting the intramedullary shaft from an end of the long bone having the epiphyseal fracture and along a length of the medullary canal.

Feature 47. The method according to any one of features 41-46, wherein the nail is inserted such that the cap portion having the one or more epiphyseal bone fragments attached thereto is secured at the end of the long bone having the epiphyseal fracture to provided fixation of the epiphyseal fracture.

Feature 48. The method according any one of features 41-47, further comprising inserting one or more self-drilling screws through an outer surface of the diaphysis of the long bone and into the intramedullary shaft of the intramedullary nail to secure the intramedullary nail having the one or more epiphyseal bone fragments attached thereto.

Feature 49. The method according to any one of features 41-48, further comprising applying a bone cement such as polymethyl methacrylate to the intramedullary shaft prior to inserting the distal end of the intramedullary nail into the medullary canal.

Feature 50. The method according to any one of features 41-49, wherein the one or more epiphyseal bone fragments comprise two or more bone fragments.

Feature 51. The method according to any one of features 41-50, wherein the epiphyseal fracture is selected from the group consisting of a Salter Harris type 1 fracture, a Salter Harris type 2 fracture, a Salter Harris type 3 facture, and a Salter Harris type 4 fracture.

Feature 52. The method according to any one of features 41-51, wherein the fracture is comminuted.

Feature 53. The method according to any one of features 41-52, wherein the patient is a human and the long bone is a radius, and wherein the epiphyseal fracture is selected from the group consisting of a Mason type II radial head fracture and a Mason type III radial head fracture.

1. An intramedullary nail for fixation of an epiphyseal fracture in a long bone of a patient in need thereof, the intramedullary nail comprising:
an intramedullary shaft configured for insertion into a medullary canal of the long bone, the intramedullary shaft having a longitudinal axis, a proximal end, and a distal end;
a cap portion at the proximal end and configured for attaching thereto one or more epiphyseal bone fragments from the epiphyseal fracture; and
a tip portion at the distal end and configured for insertion into the medullary canal of the long bone.

2-4. (canceled)

5. The intramedullary nail according to claim 4, wherein the intramedullary shaft is configured such that, when the intramedullary shaft is inserted along the length of the medullary canal, the intramedullary shaft secures the cap portion at the end of the long bone having the epiphyseal fracture.

6. The intramedullary nail according to claim 5, wherein the cap portion is configured such that, when the one or more epiphyseal bone fragments are attached to the cap portion and the cap portion is secured at the end of the long bone having the epiphyseal fracture, the intramedullary nail provides for fixation of the epiphyseal fracture.

7-13. (canceled)

14. The intramedullary nail according to claim 1, wherein the intramedullary shaft has an outer surface having a plurality of threads on the outer surface;
wherein each of the threads in the plurality of threads have an outer diameter of about 1 mm to about 2 mm;
wherein each of the threads in the plurality of threads have a height of about 0.5 mm to about 3 mm; and
wherein the plurality of threads have a number of thread starts from about 2 to 8.

15-17. (canceled)

18. The intramedullary nail according to claim 1, wherein the intramedullary shaft has a height in the longitudinal direction of about 15 mm to about 25 mm; and
wherein the intramedullary shaft has an outer diameter of about 4 mm to about 8 mm.

19. (canceled)

20. The intramedullary nail according to claim 1, wherein the intramedullary nail is made partially or entirely from a thermoplastic polymer.

21-23. (canceled)

24. The intramedullary nail according to claim 20, wherein the thermoplastic polymer has been modified to increase surface hydrophobicity.

25. (canceled)

26. The intramedullary nail according to claim 1, wherein the cap portion is configured to receive one or more self-drilling screws for attaching the one or more epiphyseal bone fragments to the cap portion.

27. The intramedullary nail according to claim 26, wherein the cap portion comprises an outer surface substantially parallel to the longitudinal axis of the intramedullary shaft, and
wherein the outer surface comprises a plurality of grooves running longitudinally along the outer surface.

28. (canceled)

29. The intramedullary nail according to claim 26, wherein the grooves on the outer surface of the cap have a depth of about 0.3 mm to about 0.9 mm.

30. The intramedullary nail according to claim 26, wherein the plurality of grooves on the outer surface of the cap comprise about 15 to 25 grooves equally spaced circumferentially along the outer surface of the cap.

31. The intramedullary nail according to claim 26, wherein the cap portion further comprise an upper surface having an opening dimensioned to receive a tip of a tool for tightening the intramedullary nail into the medullary canal.

32. The intramedullary nail according to claim 26, wherein the cap portion has a height in the longitudinal direction of about 6 mm to about 10 mm.

33. The intramedullary nail according to claim 26, wherein the cap portion has an outer diameter of about 6 mm to about 15 mm

34. The intramedullary nail according to claim 1, wherein the intramedullary shaft comprises one or more void regions sized and positioned to contain one or more shavings from a self-drilling screw being inserted into the intramedullary shaft.

35-39. (canceled)

40. The intramedullary nail according to claim 1, wherein the patient is a human patient and the long bone is a radius.

41. (canceled)

42. A method of fixation of an epiphyseal fracture in a long bone of a patient in need thereof, the method comprising:
attaching one or more epiphyseal bone fragments from the epiphyseal fracture to the cap portion of an intramedullary nail according to claim 1, and
inserting the distal end of the intramedullary nail into a medullary canal of the long bone.

43-48. (canceled)

49. The method according to claim 42, further comprising applying a bone cement such as polymethyl methacrylate to the intramedullary shaft prior to inserting the distal end of the intramedullary nail into the medullary canal.

50. (canceled)

51. The method according to claim 42, wherein the epiphyseal fracture is selected from the group consisting of a Salter Harris type 1 fracture, a Salter Harris type 2 fracture, a Salter Harris type 3 facture, and a Salter Harris type 4 fracture.

52. (canceled)

53. The method according to claim 41, wherein the patient is a human and the long bone is a radius, and
wherein the epiphyseal fracture is selected from the group consisting of a Mason type II radial head fracture and a Mason type III radial head fracture.
